(19) United States
(12) Patent Application Publication
(10) Pub. No.: US 2025/0037799 A1
(43) Pub. Date: Jan. 30, 2025

(54) DEVICE AND METHOD FOR PROVIDING INTERFACE FOR SETTING PARAMETERS

(71) Applicant: SEEGENE, INC., Seoul (KR)

(72) Inventors: Ji Hoon PARK, Gyeonggi-do (KR); Dong Jun LIM, Seoul (KR)

(21) Appl. No.: 18/836,893

(22) PCT Filed: Feb. 8, 2023
(86) PCT No.: PCT/KR2023/001789

§ 371 Date: Aug. 8, 2024

(30) Foreign Application Priority Data
Feb. 9, 2022 (KR) 10-2022-0016907

(51) Int. Cl. G16B 40/10; G16B 45/00

(52) U.S. Cl. CPC G16B 40/10; G16B 45/00

(57) ABSTRACT

According to an embodiment of the disclosure, a method for providing an interface for setting parameters, for a target analyte positive/negative result-determination process: the method comprising: displaying correct answer data for the positive/negative of the target analyte in a sample: displaying a pre-determined default value for a parameter used in the determination process: displaying a first determination result of the determination process to which the default value has been applied: receiving a setting value for the parameter; and displaying a second determination result of the determination process to which the setting value has been applied; wherein the determination process analyzes a signal obtained by a reaction of amplifying a nucleic acid as the target analyte.

### TECHNICAL FIELD

The disclosure relates to a technology for providing an interface used for setting values for parameters used in a process for determining the positive/negative of a target analyte in a sample.

### BACKGROUND ART

A target analyte, particularly target nucleic acid molecules, may be amplified by various methods: polymerase chain reaction (PCR), ligase chain reaction (LCR) (U.S. Pat. Nos. 4,683,195 and 4,683,202; PCR Protocols: A Guide to Methods and Applications (Innis et al., eds, 1990)), strand displacement amplification (SDA)) (Walker, et al. Nucleic Acids Res. 20 (7): 1691-6 (1992); Walker PCR Methods Appl 3 (1): 1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350 (6313): 91-2 (1991)), rolling circle amplification (RCA) (Lisby, Mol. Biotechnol. 12 (1): 75-99 (1999); Hatch et al., Genet. Anal. 15 (2): 35-40 (1999)) and Q-beta Replicase) (Lizardi et al., BiolTechnology 6: 1197 (1988)), loop-mediated isothermal amplication (LAMP, Y. Mori, H. Kanda and T. Notomi, J. Infect. Chemother., 2013, 19, 404-411), recombinase polymerase amplication (RPA, J. Li, J. Macdonald and F. von Stetten, Analyst, 2018, 144, 31-67).

Among nucleic acid amplification reactions, the real-time PCR system is a technology for detecting target nucleic acids in a sample in real-time. To detect a specific target nucleic acid, the real-time PCR system uses a signal generator that emits a detectable fluorescent signal in proportion to the amount of the target nucleic acid. Specifically, the fluorescent signal generated by the above-described signal generator in proportion to the amount of the target nucleic acid is detected per cycle. A data set including each measurement point and the strength of the fluorescent signal at the measurement point is obtained. An amplification curve or amplification profile curve which indicates the strength of the fluorescent signal detected relative to the measurement point from the so-obtained data set is obtained.

A process for determining the positive/negative of a specific target nucleic acid in the sample based on the above-described data set, amplification curve, or amplification profile curve is performed. The positive/negative determining process is an analysis method for determining whether a meaningful target analyte amplification is present in the data obtained from the real-time PCR detection system and requires parameters for determining, e.g., the signal value (e.g., RFU value) in a specific cycle and the cycle threshold (Ct) value for each sample, when analyzing the data generated in the reaction wells of the plate as a criterion for determining the positive/negative.

### DISCLOSURE OF INVENTION

### Technical Problem

The above-described, predetermined threshold value serves as a parameter used in the process for determining the positive/negative of the target analyte in the sample and affects the result of the positive/negative determination process. In other words, the above-described threshold is an example criterion for determining the positive/negative of the target analyte in the sample.

An embodiment of the disclosure provides an interface used for setting a value for the criterion for determining the positive/negative of the target analyte in the sample.

In the process for determining the positive/negative of the target analyte in the sample, a correction task may be performed on the above-described data set, amplification curve, or amplification profile curve. The correction task may use a predetermined parameter. The parameter value also affects the above-described result of positive/negative determination process.

An embodiment of the disclosure provides an interface used for setting a value for the parameter used in the above-described correction task.

Objects of the disclosure are not limited to the foregoing, and other unmentioned objects would be apparent to one of ordinary skill in the art from the following description.

### Solution to Problem

According to an embodiment of the disclosure, a method for providing an interface for setting parameters for a target analyte positive/negative result-determination process; the method comprising:
displaying correct answer data for the positive/negative of the target analyte in a sample, displaying a pre-determined default value for a parameter used in the determination process, displaying a first determination result of the determination process to which the default value has been applied, receiving a setting value for the parameter, and displaying a second determination result of the determination process to which the setting value has been applied; wherein the determination process analyzes a signal obtained by a reaction of amplifying a nucleic acid as the target analyte.

The method further comprises providing a section for displaying the first determination result and the second determination result, wherein the first determination result and second determination result are together displayed in the section for displaying the determination results.

The correct answer data is displayed in the section for displaying the determination results.

The method further comprises providing a section for displaying a value of the parameter. Wherein the default value and the setting value are displayed in the section for displaying the value of the parameter.

The method further comprises receiving a changed value for the setting value and displaying the second determination result of the determination process to which the changed value has been applied.

The method further comprises displaying, on one screen, the setting value, the changed value and determination results by using the setting value and changed value.

The method further comprises providing on one screen, both (i) a section for displaying the first and second determination results of the determination process and (ii) a section for displaying the pre-determined default value and the setting value of the parameter.

The method selecting any one of the at least two values comprising the setting value and one or more changed setting values; and displaying the determination result of the determination process to which the selected value has been applied.

The parameter is used in mathematical processing of the signal and/or is applied to the signal to be used as a criterion for determining the positive/negative of the target analyte in the sample.

The criterion includes a threshold intensity of the signal used to determine a threshold cycle (Ct) of the sample.

The parameter includes a reference value for correcting a signal dependent upon the presence of the target analyte in the sample, the signal value relation parameter (cut-off ratio (CR)) reflecting a rule of a signal change at different detection temperatures, a parameter criterion (PMC) regarding determination of a shape of a sigmoid graph for a data set obtained through the signal generation reaction, a start fitting cycle (SFC) for correcting a baseline fitting start cycle, a minimum fitting cycle (MFC) used in baseline fitting from the SFC to a minimum MFC to correct the data set, and a determinant RFU (dRFU) regarding displacement of a data set obtained from the signal generation response for the positive/negative of the target analyte.

The determination process is performed using a result of a reaction performed using a plate including a plurality of reaction wells containing the target analyte. The method further comprises displaying a position of a reaction well different from the correct answer data of the determination result of the determination process to which the default value is applied, on an imaged plate and displaying a position of a reaction well different from the correct answer data of the determination result of the determination process to which the setting value is applied, on an imaged plate.

The method further comprises displaying amplification property data when the reaction well is selected.

The method further comprises displaying the received setting value and the amplification property data on one screen.

The method further comprises displaying amplification property data corresponding to a determination result different from the correct answer data of the first determination result of the determination process to which the default value is applied or displaying amplification property data corresponding to a determination result different from the correct answer data of the second determination result of the determination process to which the setting value is applied.

The method further comprises displaying the default value and the amplification property data on one screen or displaying the received setting value and the amplification property data on one screen.

According to an embodiment, a device for providing an interface for setting parameters comprises a memory, a processor, and an interface providing unit driven by the processor. The interface providing unit is implemented to be driven by the processor executing instructions stored in the memory to display correct answer data for the positive/negative of a target analyte in a sample, display a pre-determined default value for a parameter used in a positive/negative determination process, display a determination result of the determination process to which the default value is applied, receive a setting value for the parameter, and display a determination result of the determination process to which the setting value is applied.

### Advantageous Effects of Invention

According to an embodiment, a researcher/developer of a reagent for determining the positive/negative of the target analyte in the sample may be provided with default values for parameters used in the process of determining the positive/negative of the target analyte in the sample as a reference. By referring to such a default value, the developer may quickly and accurately set parameter values that lead to the same results as the correct answer about the positive/negative, i.e., positive or negative, as much as possible.

Further, the researcher/developer may compare/contrast the positive/negative determination results according to the above-described default value and the correct answer data with the positive/negative determination results according to parameter values when they enter the parameter values.

### BRIEF DESCRIPTION OF DRAWINGS

The above and other objects, features, and advantages of the disclosure will be more clearly understood from the following detailed description, taken in conjunction with the accompanying drawings, in which:

FIG. 1 is a view illustrating a detection device 200 and a parameter setting interface providing device 100 according to an embodiment;

FIG. 2 is a block diagram illustrating a parameter setting interface providing device according to an embodiment;

FIG. 3 is a view illustrating an example screen providing a parameter setting interface for performing a parameter setting interface providing method according to an embodiment; and

FIG. 4 is a flowchart illustrating a parameter setting interface providing method according to an embodiment.

### MODE FOR THE INVENTION

Advantages and features of the disclosure, and methods for achieving the same may be apparent from the embodiments described below with reference to the accompanying drawings. However, the disclosure is not limited to the embodiments disclosed herein, and various changes may be made thereto. The embodiments disclosed herein are provided only to inform one of ordinary skilled in the art of the category of the disclosure. The disclosure is defined only by the appended claims.

When determined to make the subject matter of the disclosure unclear, the detailed description of known functions or configurations may be skipped. The terms described below are defined considering the functions in embodiments of the disclosure and may be replaced with other terms according to the intention or practice of the user or operator. Therefore, the terms should be defined based on the overall disclosure.

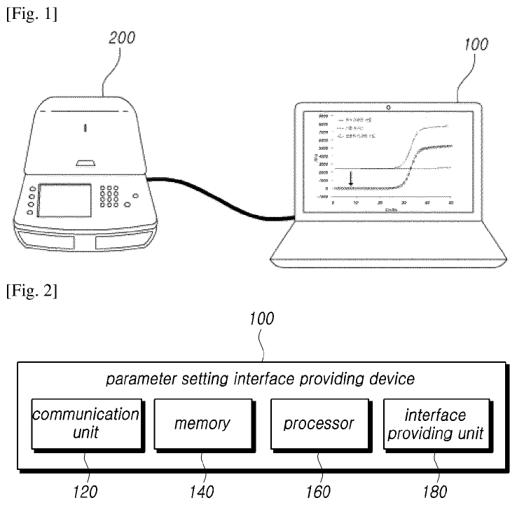

Prior to describing FIG. 1, terms used herein are described.

In the disclosure, the term “nucleic acid amplification reaction” includes various PCRs based on polymerase chain reaction. For example, quantitative PCR, digital PCR, asymmetric PCR, reverse transcriptase PCR (RT-PCR), differential display PCR (DD-PCR), nested PCR, arbitrary priming PCR (AP-PCR), multiplex PCR, SNP genome typing PCR and the like are included.

In the disclosure, the term “plate” refers to a standard unit in which an amplification reaction is performed in a PCR device, and a basic unit in which data generated after an amplification reaction is stored. Different plates may be plates where amplification reactions are performed at different times using the same amplification device or plates where amplification reactions are performed by different amplification devices at the same time.

The plate includes a plurality of reaction wells. The plate may include N×M reaction wells. The plate typically includes 12×8 or 8×12 reaction wells. The reaction well of the plate may be shaped as a tube that is integrated or separable from the plate. The plate may have a rectangular shape. The plate may include one or more reaction wells and may be implemented in various shapes, such as a circle, a trapezoid, or a rhombus, as well as a rectangle.

The well of the plate contains the sample to be analyzed and the reagents necessary for the nucleic acid amplification reaction.

The term “target analyte” may include a variety of substances (e.g., biological and non-biological substances), which may refer to the same target as the term “target analyte”

Specifically, the target analyte may include a biological substances, more specifically at least one of nucleic acid molecules (e.g., DNA and RNA), proteins, peptides, carbohydrates, lipids, amino acids, biological compounds, hormones, antibodies, antigens, metabolites, and cells.

The term “sample” includes biological samples (e.g., cells, tissues, and body fluids) and non-biological samples (e.g., food, water, and soil). Among them, the biological sample may include at least one of, e.g., virus, germs, tissues, cells, blood (including, e.g., whole blood, plasma, and serum), lymph, bone marrow fluid, saliva, sputum, swab, aspiration, milk, urine, stool, ocular humor, semen, brain extracts, spinal fluid, joint fluid, thymus fluid, bronchoalveolar lavage fluid, ascites, and amniotic fluid. Such sample may or may not include the above-described target analyte.

When the above-described target analyte is or includes a nucleic acid molecule, a nucleic acid extraction process as known in the art may be performed on the sample estimated to contain the target analyte. (See Sambrook, J. et al., Molecular Cloning. A Laboratory Manual, 3rd ed. Cold Spring Harbor Press (2001)). The nucleic acid extraction process may vary depending on the type of sample. Further, when the extracted nucleic acid is RNA, a reverse transcription process may be additionally performed to synthesize cDNA (See Sambrook, J. et al., Molecular Cloning. A Laboratory Manual, 3rd ed. Cold Spring Harbor Press (2001).

The term “data set” refers to the data obtained from a signal generation reaction on the target analyte using a signal generator (the signal generator is described below).

In the case, the term “signal generation reaction” refers to a reaction that generates a signal depending on the properties of the target analyte in the sample, such as activity, amount or presence (or absence), specifically presence (or absence). The signal generation reactions include biological reactions and chemical reactions. Among them, the biological reactions include genetic analysis processes, such as PCR, real-time PCR and microarray analysis, immunological analysis processes, and bacterial growth analysis. Further, the chemical reactions include the process of analyzing the creation, change or destruction of chemicals. According to an embodiment, the signal generation reaction may be a genetic analysis process or may be nucleic acid amplification reaction, enzymatic reaction or microbial growth.

The above-described signal generation reactions are accompanied by signal changes. Therefore, the progress of the signal generation reaction may be evaluated by measuring the change in signal.

Here, the term “signal” means a measurable output. Further, the measured magnitude or change in the signal serves as an indicator qualitatively or quantitatively indicating the properties of the target analyte, specifically, the positive/negative of the target analyte in the sample.

Here, examples of the indicator include fluorescence intensity, luminescence intensity, chemiluminescence intensity, bioluminescence intensity, phosphorescence intensity, charge transfer, voltage, current, power, energy, temperature, viscosity, light scatter, radioactivity intensity, reflectance, transmittance and absorbance, but are not limited thereto.

The term “signal generator” as described above means a means for providing a signal indicative of the properties of the target analyte to be analyzed, specifically the positive/negative of the target analyte.

The signal generator includes a label itself or a label-attached oligonucleotide.

The label includes a fluorescent label, a luminescent label, a chemiluminescent label, an electrochemical label, and a metal label. The label may be used as a label itself, such as an intercalating dye. Or, the label may be used as a single label or interactive dual label including a donor molecule and an acceptor molecule, in the form attached to one or more oligonucleotides.

When a fluorescent label is used, the signal value may be represented as a relative fluorescence unit (RFU) value.

The signal generator may include an enzyme with nucleolytic activity to generate signals (e.g., an enzyme with 5′ nucleolytic activity or an enzyme with 3′ nucleolytic activity).

Various methods are known for generating a signal indicating the presence of a target analyte, particularly a target nucleic acid molecule, by the signal generator. Representative examples may include: TaqMan™ probe method (U.S. Pat. No. 5,210,015), molecule beacon method (Tyagi, Nature Biotechnology v.14 Mar. 1996), Scorpion method (Whitcombe et al., Nature Biotechnology 17:804-807 (1999)), Sunrise or Amplifluor method (Nazarenko et al., Nucleic Acids Research, 25 (12): 2516-2521 (1997), and U.S. Pat. No. 6,117,635), Lux method (U.S. Pat. No. 7,537,886), CPT (Duck P, et al. Biotechniques, 9:142-148 (1990)), LNA method (U.S. Pat. No. 6,977,295), Plexor method (Sherrill C B, et al., Journal of the American Chemical Society, 126:4550-4556 (2004)), Hybeacons (D. J. French, et al., Molecular and Cellular Probes 13:363-374 (2001) and U.S. Pat. No. 7,348,141), Dual-labeled, self-quenched probe; U.S. Pat. No. 5,876,930), hybridization probe (Bernard P S, et al., Clin Chem 2000, 46, 147-148), PTOCE (PTO cleavage and extension) method (WO 2012/096523), PCESH (PTO Cleavage and Extension-Dependent Signaling Oligonucleotide Hybridization) method (WO 2013/115442), PCE-NH (PTO Cleavage and Extension-Dependent NonHybridization) method (PCT/KR2013/012312) and CER method (WO 2011/037306).

The above-described term “signal generation reaction” may include a signal amplification reaction. In this case, the term “amplification reaction” means a reaction that increases or decreases the signal generated by the signal generator. Specifically, the amplification reaction refers to a reaction for increasing (or amplifying) the signal generated by the signal generator depending on the presence of the target analyte.

The amplification reaction may or may not be accompanied by amplification of the target analyte (e.g., nucleic acid molecule). More specifically, the amplification reaction may refer to a signal amplification reaction accompanied by amplification of the target analyte.

The data set obtained through the amplification reaction may include an amplification cycle.

The term “cycle” refers to a unit of change in a predetermined condition in a plurality of measurements accompanied by the change in the condition. The change in the predetermined condition means, e.g., an increase or decrease in temperature, reaction time, number of reactions, concentration, pH, or number of copies of the measurement target (e.g., nucleic acid). Thus, the cycle may be a time or process cycle, unit operation cycle, or reproductive cycle.

More specifically, the term “cycle” may mean one unit of repetition when a reaction of a predetermined process is repeated or a reaction is repeated based on a predetermined time interval.

For example, in the case of polymerase chain reaction (PCR), one cycle means a reaction including denaturation of nucleic acids, annealing of primers, and extension of primers. In this case, the change in the predetermined condition is an increase in the number of repetitions of the reaction, and a repeating unit of the reaction including the above series of steps is set as one cycle.

Alternatively, the term “cycle” may mean one unit of repetition when a predetermined action is repeated as the reaction proceeds.

For example, when a nucleic acid amplification reaction is performed, the act of detecting a signal generated at regular time intervals may be repeated and may mean one unit of the repetition. In this case, the cycle may have a unit of time. The amplification reaction for amplifying the signal indicating the presence of the target analyte may be performed in a manner in which the signal is also amplified while the target analyte is amplified (e.g., real-time PCR method). Alternatively, according to an embodiment, the amplification reaction may be performed in a manner in which only a signal indicating the presence of the target analyte is amplified without amplifying the target analyte (e.g., CPT method (Duck P, et al., Biotechniques, 9:142-148 (1990)), Invader assay (U.S. Pat. Nos. 6,358,691 and [symbol] 6, 194, 149).

The above-described target analyte, particularly target nucleic acid molecules, may be amplified by various methods: polymerase chain reaction (PCR), ligase chain reaction (LCR) (U.S. Pat. Nos. 4,683,195 and 4,683,202; PCR Protocols: A Guide to Methods and Applications (Innis et al., eds, 1990)), strand displacement amplification (SDA)) (Walker, et al. Nucleic Acids Res. 20 (7): 1691-6 (1992); Walker PCR Methods Appl 3 (1): 1-6 (1993)), transcription-mediated amplification (Phyffer, et al., J. Clin. Microbiol. 34:834-841 (1996); Vuorinen, et al., J. Clin. Microbiol. 33:1856-1859 (1995)), nucleic acid sequence-based amplification (NASBA) (Compton, Nature 350 (6313): 91-2 (1991)), rolling circle amplification (RCA) (Lisby, Mol. Biotechnol. 12 (1): 75-99 (1999); Hatch et al., Genet. Anal. 15 (2): 35-40 (1999)) and Q-Beta Replicase) (Lizardi et al., BiolTechnology 6: 1197 (1988)), loop-mediated isothermal amplication (LAMP, Y. Mori, H. Kanda and T. Notomi, J. Infect. Chemother., 2013, 19, 404-411), recombinase polymerase amplication (RPA, J. Li, J. Macdonald and F. von Stetten, Analyst, 2018, 144, 31-67).

The amplification reaction amplifies the signal while accompanying the amplification of the target analyte (specifically, the target nucleic acid molecule). For example, the amplification reaction is carried out by PCR, specifically real-time PCR, or isothermal amplification reaction (e.g., LAMP or RPA).

The data set obtained by the signal generation reaction include a plurality of data points including cycles of the signal generation reaction and signal values in the cycles.

The term “signal value” refers to a value obtained by quantifying the signal level (e.g., signal intensity) actually measured in a cycle of a signal generation reaction, in particular, an amplification reaction, according to a predetermined scale, or a changed value thereof. The changed value may include a mathematically processed signal value of the actually measured signal value. Examples of mathematically processed signal values of actually measured signal values (i.e., signal values of raw data sets) may include logarithmic values or derivatives.

The term “data point” means a coordinate value including the cycle and signal value. Further, the term “data” refers to all the information constituting the data set. For example, each cycle and signal value of an amplification reaction may correspond to data.

Data points obtained by a signal generation reaction, particularly amplification reaction may be displayed with coordinate values that may be shown in the two-dimensional rectangular coordinate system. In the coordinate values, the X axis denotes the number of cycles, and the Y axis denotes the signal value measured or processed in the cycle.

The term “data set” means a set of the data points. For example, the data set may be a set of data points obtained directly through the amplification reaction performed in the presence of the signal generator or a changed data set of such a data set. The data set may be all or some of a plurality of data points obtained by the amplification reaction or changed data points thereof.

The data set may be a data set obtained by processing a plurality of data sets. When analysis on a plurality of target analytes is performed in one reaction vessel, the data set for the plurality of target analytes may, in some cases, be obtained by processing the data sets obtained from the reaction performed in the one reaction vessel. For example, the data set for the plurality of target analytes in the one reaction vessel may be obtained by processing the plurality of data sets obtained from the signals measured at different temperatures.

The above-described data set may be floated, and an amplification curve may thereby be obtained.

Various embodiments of the disclosure are described below with reference to the drawings.

FIG. 1 is a view illustrating a detection device 200 and a parameter setting interface providing device 100 according to an embodiment. The devices 100 and 200 may be connected to each other by wired or wireless communication. However, the block diagram of FIG. 1 is merely exemplary, and the spirit of the disclosure is not limited to those shown in FIG. 1. For example, additional components not shown in FIG. 1 may be connected to the devices 100 and 200. Alternatively, unlike shown, the parameter setting interface providing device 100 may be implemented to be included in the detection device 200. However, it is assumed that the above-described components 100 and 200 are implemented or connected as shown in FIG. 1. Each component is described below in detail.

The detection device 200 is implemented to perform a nucleic acid amplification reaction and a nucleic acid detection task on the sample. According to an embodiment, the detection device 200 may be implemented to perform the nucleic acid detection task without performing the nucleic acid amplification reaction, but the following description assumes that the detection device 200 is implemented to perform the nucleic acid amplification reaction as well.

As the above-described nucleic acid amplification reaction is performed, the amount of a target analyte, if contained in the sample in the reaction vessel, may be amplified, and the magnitude of the signal generated by the above-described signal generator may be amplified as well. The detection device 200 is implemented to detect the magnitude of the signal. Specifically, the detection device 200 may monitor, in real-time, the magnitude of the signal which is changed as the nucleic acid amplification reaction proceeds. The monitoring result may be output from the detection device 200, in the form of a data set as mentioned in the term definition section, e.g., as the magnitude of signal per cycle. Here, the intensity of signal per cycle is information as material that is a basis for determining the positive/negative of the target analyte in the sample.

In the sense that if the nucleic acid amplification reaction is performed, the magnitude of the signal generated by the signal generator may also be amplified, the nucleic acid amplification reaction is regarded below as causing a signal generation reaction as described above in the terminology section.

The parameter setting interface providing device 100 provides a user interface for setting parameter values. This is described below in detail.

Various types of software may be used in the process for determining the positive/negative of the target analyte in the sample. Among such software, there is positive/negative determination software that performs the process for determining the positive/negative of the target analyte in the sample, using the parameters whose values are predetermined if information is input as the above-described material. The performance or accuracy of the positive/negative determination software is dependent upon the parameter value. The quicker the parameter value is determined, the earlier the software and the reagent associated therewith may be released.

According to an embodiment, the parameter setting interface providing device 100 is implemented to provide a user interface for reagent researchers/developers to make it possible to quickly and accurately determine the parameter values.

The above-described parameter operates as a criterion for determining the positive/negative in the process for determining the positive/negative of the target analyte in the sample. As such criteria, various parameters may be included and, among them, one parameter is threshold which is described below.

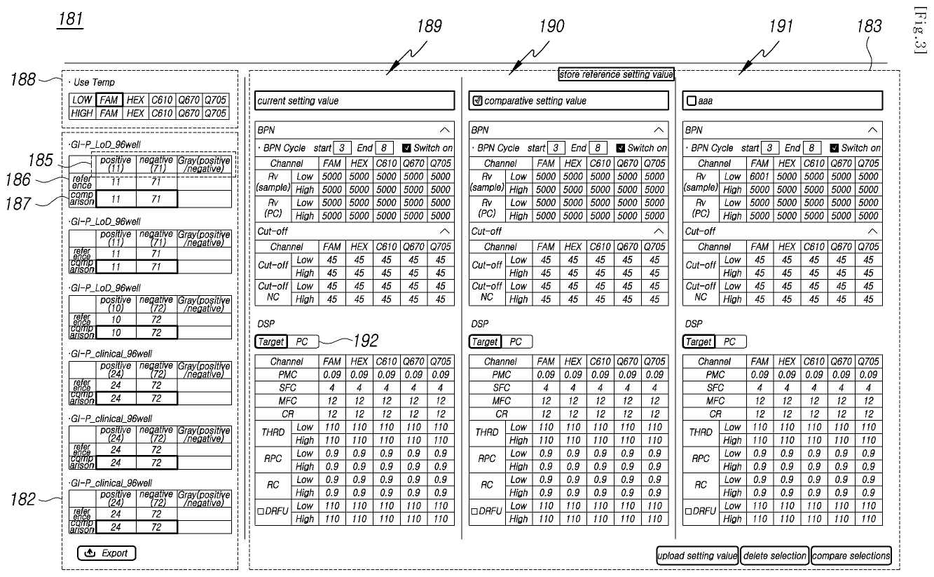

Each functional component of the parameter setting interface providing device 100 is described below in detail with reference to FIG. 2.

FIG. 2 is a block diagram illustrating a parameter setting interface providing device 100 according to an embodiment. The parameter setting interface providing device 100 may be implemented on a laptop computer, server, or cloud, but is not limited thereto.

Referring to FIG. 2, the parameter setting interface providing device 100 includes a communication unit 120, a memory 140, a processor 160, and an interface providing unit 180, but is not limited thereto.

The communication unit 120 is implemented as a wired or wireless communication module. The parameter setting interface providing device 100 may perform communication with the outside through the communication unit 120. For example, the parameter setting interface providing device 100 may transmit the setting value received by the researcher/developer to the outside through the communication unit 120 as described below. As described below, the parameter setting interface providing device 100 may receive, e.g., correct answer data, default value, a first determination result by the determination process when the default value is applied, and a second determination result by the determination process when the setting value is applied, from the outside through the communication unit 120.

The memory 140 stores various types of data. The stored data may include the above-mentioned correct answer data or default value, but is not limited thereto. For example, the memory 140 may store the data (or data set) received from the detection device 200 or the data processed by the processor 160, which may include the above-described data set, noise-canceled data set, amplification curve, Ct value, or signal value (e.g., RFU value).

The memory 140 may store the data in units of plates or units of strips. The memory 140 may store data for a plurality of plates and amplification data generated based on the data for the plurality of plates.

The data for a plate includes the number or character for identifying the plate, information recorded for the plate, or data for the reaction wells included in the plate. The information recorded for the plate includes various pieces of information, such as the date, time, or method for performing the amplification reaction on the plate. The data for reaction wells includes the data set, noise-canceled data set, amplification curve, Ct value, or signal value for each of the reaction wells.

Although FIG. 2 illustrates that the memory 140 is a component separate from the processor 160, the memory 140 and the processor 160 may be implemented as a single device. For example, the memory 140 may be storage, such as a cache included in the processor 160.

The processor 160 may be implemented by a central processing unit (CPU), graphics processing unit (GPU), micro controller unit (MCU), or a dedicated processor for performing methods according to an embodiment.

The processor 160 may write data in the memory 140. The processor 160 may read and execute commands stored in the memory 140. For example, the processor 160 may execute commands stored in the memory 140 to enable the interface providing unit 180 to perform functions described below.

The interface providing unit 180 may be driven by the processor 160 as follows. The interface providing unit 180 may be implemented by a means displaying data and a means receiving data. For example, the interface providing unit 180 may be implemented as a touchscreen or touchpad or may be implemented as a combination of an LCD monitor and a keyboard.

The interface providing unit 180 displays correct answer data about the positive/negative of the target analyte in the sample. The correct answer data may be received from the outside through the communication unit 120 by the parameter setting interface providing device 100 or may be loaded from the memory 140, but is not limited thereto.

The correct answer data is a collection of correct answers in which the presence or absence of the target per sample is identified as positive or negative. When the correct answers (positive or negative) for each reaction vessel receiving the sample are identified for each 96-well plate reaction vessel, it may be said to be correct answer data for the 96-well plate. The correct answer data may be labeled and obtained per well by the researcher/developer who herself has adjusted the concentration of the target nucleic acid for each reaction vessel.

In this case, the target nucleic acid may be any one of an artificially synthesized nucleic acid, a standard strain or an actual clinical specimen.

According to an embodiment, the correct answer data may be expressed as the number of positives identified as positive, number of negatives identified as negative, or number of grays identified as gray (positive or negative) in the 96-well plate (185 (positives (11), negatives (71) in the description below in connection with FIG. 3). According to an embodiment, the correct answer data may be displayed on the plate image.

For each well on the plate, the correct answer (positive, negative, or gray) may be displayed in a color (e.g., red for positive, blue for negative) for identifying positive/negative or may be displayed in an icon or text.

As such, the correct answer data may be represented in various manners.

As the correct answer data, data (positive, negative, or gray) labeled by the researcher/developer who has adjusted the concentration of the target nucleic acid for each reaction vessel as described above may be used, or a result of actually determining positive or negative from an actual clinical experiment (e.g., amplification reaction) may be used, but is not limited thereto.

The disclosure may display the parameter value to converge to the correct answer for each well, by applying a positive/negative result-determination process to the data set obtained through the amplification reaction on the sample providing the correct answer data and comparing the result of the application with the correct answer data.

Then, the interface providing unit 180 displays a determined default value, for the parameter used in the process for determining the positive/negative of the target analyte in the sample.

Specifically, the process for determining the positive/negative of the target analyte in the sample means determining whether the target analyte is present in the sample, based on the above-described material obtained from the detection device 200.

Here, the positive/negative is determined using a nucleic acid amplification reaction dependent upon the target analyte, and the positive/negative determination process includes a process for analyzing the signal obtained by the nucleic acid amplification reaction with the target analyte. The positive/negative determination process according to the disclosure requires parameters for determining positive/negative, for determining whether each sample is positive or negative using the data set obtained for each sample providing correct answer data. For example, the parameters used in the positive/negative determination process may include the threshold intensity of the signal used to determine the threshold cycle (Ct) for each sample as a criterion for determining the positive/negative of the target analyte in the sample.

Accordingly, the correct answer data means the determining result identified as positive or negative for the target analyte for each sample, for the Ct parameter.

The parameters are described below. The parameters are operated as criteria for determining the positive/negative in the sense that they affect the results by the positive/negative result-determination process. The parameters may include threshold, but are not limited thereto. As a parameter, the threshold is used when compared with the signal intensity per cycle, as the above-described material, in the above-described positive/negative determination software. This is described in detail. The number of the cycle reaching or exceeding the threshold among the per-cycle signal intensities is detected by the above-described positive/negative determination software. The detected cycle number is assigned as the Ct value. The positive/negative result-determination process determines negative if the assigned Ct value is larger than a reference Ct value or negative if smaller than the reference Ct value. The method for determining positive/negative by the Ct value is not limited thereto.

The default value is described below. The default value refers to a reference value provided as default for the parameter. For example, the default value for the threshold may be 3000 RFU or 4100 RFU, but is not limited thereto.

The default value may be determined in various manners. For example, the default value may be input in advance by a master operator operating the parameter setting interface providing device 100. In contrast, the default value may be determined in advance by deep learning or statistical method.

In some cases, such a default value may be categorized and its value determined in advance. For example, categories may be divided into default values for respiratory products, default values for intestinal bacteria, and default values for sexually transmitted diseases, and a specific default value may be present for each category.

The default value whose value is determined may be loaded from the memory 140 or loaded from the outside through the communication unit 120.

The interface providing unit 180 displays the determination result by the positive/negative result-determination process when the parameter value is the above-described default value.

The determination result by the positive/negative result-determination process when the parameter value is the above-described default value may be determined by the above-described positive/negative determination software. Alternatively, the determination result by the positive/negative determination process may be one determined by executing an instruction stored in the memory 140 by the processor 160 shown in FIG. 2.

The determination result may include the number of ‘positives’ and the number of ‘negatives’ like the correct answer data and, in some cases, may include the number of ‘grays’.

In the disclosure, the determination result may include a first determination result and a second determination result.

The first determination result to which the default value has been applied.

The second determination result to which the setting value has been applied.

The interface providing unit 180 receives a setting value for the parameter. In other words, the setting value is a value that is supposed to be input. The entity that performs the input is the above-described reagent researcher/developer.

The interface providing unit 180 displays the second determination result of the determination process to which the setting value is applied.

The second determination result by the positive/negative determination process when the parameter value is the above-described setting value may be determined by the above-described positive/negative determination software. Alternatively, the determination result by the positive/negative determination process may be one determined by executing an instruction stored in the memory 140 by the processor 160 shown in FIG. 2.

The determination result may include the number of ‘positives’ and the number of ‘negatives’ like the correct answer data and, in some cases, may include the number of ‘grays’.

In other words, as described above, the reagent researcher/developer may refer to the following information, as a reference, displayed by the interface providing unit 180 of the parameter setting interface providing device 100 when inputting the parameter value used in the positive/negative determination software.
Correct answer data
Default value
The first determination result by determination process to which the default value is applied

Accordingly, the reagent researcher/developer may quickly estimate the setting value that may be closest to the correct answer data by considering the default value and the resultant the first determination result.

Further, the second determination result of the determination process for the setting value input by the researcher/developer of reagent may also be identified along with the information that may be referred to as the reference. In other words, since the researcher/developer may compare/contrast the feedback for the setting value that they themselves have input with the correct answer data or the first determination result for the default value, accelerating the process of finding the optimized setting value.

An implementation of providing an interface through the interface providing unit 180 in the parameter setting interface providing device 100 according to the disclosure is described below in detail in conjunction with an actual screen. Specific examples not specifically mentioned above may be described for the actual screen, and such examples may also be likewise applied to the foregoing description.

FIG. 3 is a view illustrating an example screen providing a parameter setting interface for performing a parameter setting interface providing method according to an embodiment.

Referring to FIG. 3, a parameter setting interface providing device 100 provides a display 181 that displays a section 182 for displaying a determination result of the determination process and a section 183 for displaying values, through the interface providing unit 180.

The determination result of applying each of the default value and setting value to the first determination process and the second determination process and analyzing them is displayed in the determination result display section 182.

According to an embodiment, the default value and the setting value are a default value and setting value for Ct.

The correct answer data 185 is displayed in the determination result display section 182.

The parameter setting interface providing device 100 according to the disclosure displays the default value-applied the first determination process determination result 186 and the setting value-applied the second determination process determination result 187 together in one table 184 in the determination result display section 182 to be compared together by the researcher/developer using the parameter setting interface providing device 100. According to an embodiment, correct answer data may further be displayed in the determination result display section.

According to an embodiment, one or more 96-well plates are prepared as reagents for detecting the same target nucleic acid. Correct answer data 185 identified as positive, negative, or gray for each sample including the correct answer labeled by the researcher/developer and obtained per well, adjusted for the concentration of the target nucleic acid for each reaction vessel or the correct answer for the result of performing nucleic acid amplification reaction on the samples for each 96-well plate, the default value-applied the first determination result 186, and the setting value-applied the second determination result 187 may be arranged and displayed in the table. Alternatively, as reagents for detecting different target nucleic acids, at least two or more 96-well plates are prepared. The correct answer data 185 identified for samples for each 96-well plate, the default value-applied the first determination result 186, and the setting value-applied the second determination result 187 may be arranged and displayed in the table 184.

The table 184 may be created for each plate, so that at least one or more per-plate tables may be displayed in the determination result display section 182.

Preferably, the table may be created for each plate for determining the positive/negative of different standard analytes and displays the correct answer, pre-determined default value, and setting value data for each plate.

According to an embodiment, a plurality of tables may be created for the plates for determining the positive/negative of the same standard analyte. In this case, a table for each plate is created for the type of template for each amplification reaction (artificially synthesized, standard strain, or actual clinical sample).

Since the default value-applied the first determination result 186 is pre-determined in the parameter setting interface providing device 100, and is the determination result of the determination process performing analysis according to the pre-determined default value, the table 184 may display the number of positives, the number of negatives, and gray (positive/negative) as reference items.

The reason why the default value-applied the first determination result 186 may be displayed as the reference item is because, as described above, the first determination result 186 by the determination process to which a pre-determined default value is applied needs to match the correct answer data 185 and is thus pre-determined in the parameter setting interface providing device by a skilled experimenter based on the experimental result identified by an actual clinical experiment.

Thus, the experimenter or developer who analyzes the sample using the parameter value according to the process of determining the positive/negative using the data set for the sample for which the correct answer is known may input the setting value based on the pre-determined default value, so that the pre-determined default value may serve as a reference when inputting the setting value for each parameter.

In the disclosure, since the setting value-applied the second determination result 187 is the determination result of the determination process performing analysis according to the setting value for each parameter input by the user, the table 184 may display the number of positives, the number of negatives, and gray (positive/negative).

The table 184 is merely an embodiment displayed in the determination result display section 182 according to the disclosure, but is not limited thereto.

In the determination result display section 182, the per-plate correct answer data and default value-applied the first determination result 186 and setting value-applied the second determination result 187 are arranged and displayed as described above. The determination result data is present for each detection channel.

Detection channels may be arranged and displayed in a use temp table 188 to be selected in the determination result display section 182 in the device according to the disclosure, so that the determination result data may be selectively displayed for each detection channel. Each detection channel arranged in the use temp table 188 may be rendered as a user interface to be selectable by the user, and the correct answer data and the default value- and setting value-applied determination results in the selected detection channel are displayed in the determination result display section 182. The detection channels are classified as high (high temperature) and low (low temperature). Accordingly, for example, the user may request to display the correct answer data, default value-applied determination result, and setting value-applied determination result in the low-temperature FAM channel or request to display the correct answer data, default value-applied the first determination result, and setting value-applied the second determination result in the high-temperature HEX channel.

The detection channel allows a light in a specific wavelength band to be selectively detected. Thus, only a specific optical marker among the optical markers in the sample emits an optical signal that is detected for each specific wavelength band. The optical marker may be selected from the group consisting of Cy2™, YO-PRO™-1, YOYO™-1, Calcein, FITC, FluorX™, Alexa™, Rhodamine 110, Oregon Green™ 500, Oregon Green™ 488, RiboGreen™, Rhodamine Green™, Rhodamine 123, Magnesium Green™, Calcium Green™, TO-PRO™-1, TOTO1, JOE, BODIPY530/550, Dil, BODIPY TMR, BODIPY558/568, BODIPY564/570, Cy3™, Alexa™ 546, TRITC, Magnesium Orange™, Phycoerythrin R&B, Rhodamine Phalloidin, Calcium Orange™, Pyronin Y, Rhodamine B, TAMRA, Rhodamine Red™, Cy3.5™, ROX, Calcium Crimson™, Alexa™ 594, Texas Red, Nile Red, YO-PRO™-3, YOYO™-3, R-phycocyanin, CPhycocyanin, TO-PRO™-3, TOTO3, DID DilC(5), Cy5™, Thiadicarbocyanine, Cy5.5, HEX, TET, Biosearch Blue, CAL Fluor Gold 540, CAL Fluor Orange 560, CAL Fluor Red 590, CAL Fluor Red 610, CAL Fluor Red 635, FAM, Fluorescein, FluoresceinC3, Pulsar 650, Quasar 570, Quasar 670, and Quasar 705 In particular, the optical marker may be selected from the group consisting of FAM, CAL Fluor Red 610, HEX, Quasar 670, and Quasar 705. The optical label includes a fluorescent label, a luminescent label, a chemiluminescent label, an electrochemical label, and a metal label.

In particular, the dye in the disclosure may be a fluorescent optical label selected from the group consisting of FAM, HEX, CAL Fluor Red 610, Quasar 670, and Quasar 705.

Whenever a plurality of target analytes are detected (low temperature/high temperature) through one channel, and/or when a plurality of target analytes are detected through a plurality of channels, circumferential direction, default value-applied determination result and setting value-applied determination result may be arranged and displayed in the first determination result and the second determination display section 182.

The default value and setting value each may be displayed in the value display section 183.

If a preparation space specified as a current setting value is displayed in the value display section 183, this is a default value for Ct and, as described above, the default value may be output, as a pre-determined fixed value, in the value display section 183 and displayed in the value display section 183, but may be a value that cannot be changed and is restricted for user access. The default value specified and displayed as the current setting value in the value display section 183 is a value for Ct regarding the determination result displayed as the reference item in the 211 table in the determination result display section 182. When the default value is displayed in the value display section 183, the device according to the disclosure may display the displayed default value-applied determination result of the first determination process in the table (184) reference item in the determination result display section 182. The parameter set means a group of at least one or more parameters for each of at least one or more detection channels.

According to an embodiment, the parameter set is a parameter set for setting the Ct parameter set for each detection channel.

According to an embodiment, the default value may be a value in a user input wait state in which it may be changed. Accordingly, for the default value, a value predetermined and displayed may be initialized, and in the value display section 183, a value for each parameter may be newly input by the user. When the default value for each parameter is newly input in the value display section 183, the device according to the disclosure may again display the first determination result of the determination process to which the newly input default value is applied, as the table (184) reference item in the determination result display section 182.

The default value is a value for outputting the determination result of positive, negative, and gray (positive/negative), displayed in the reference item for each table in the determination result display section 182, and default values for Ct may be grouped and displayed per detection channel by specifying, e.g., it as the current setting value in the value display section 183.

Accordingly, the user may recognize the degree of the value for each parameter of the default value pre-determined for Ct which becomes a reference of the parameter used in the determination process. As described above, since the pre-determined default value is set by a skilled experimenter based on the experimental result identified by an actual clinical experiment so that the default value-applied the first determination result of the determination process matches the correct answer data.

If the parameter set specified as a comparison setting value in the value display section 183 is displayed, this is a setting value for Ct and, as described above, the setting value is a value that is selected and input by the user. The setting value may be previously input by the user and be previously stored with a specific name in the parameter set unit and be invoked and displayed in the value display section 183.

For the invoked and displayed setting value parameter set, a changed value for the setting value for each parameter may be newly input by the user in the value display section 183.

According to an embodiment, a parameter set (not shown) in an input wait state in which per-parameter values are blank so that a value may be input for each parameter immediately in the value display section 183. In this case, the parameter set is a setting value parameter set in which the setting value is received for each parameter and may then be stored with a specific name, and is a setting value parameter set that may be invoked with the specific name.

At least one or more setting value input parameter sets may be displayed in the value display section 183. In this case, the user may check any one setting value input parameter set among the setting value input parameter sets displayed in the value display section 183 and select it as a setting value input parameter set that may be compared with the correct answer data and the pre-determined default value-applied the first determination result. The parameters of the setting value input parameter sets or per-parameter setting values are setting values that may be input (or changed) by the user. However, to display the setting value-applied the second determination result comparable with the pre-determined default value-applied the first determination result, any one setting value parameter set should be selected from among the setting value input parameter sets.

Accordingly, one or more setting values input parameter sets may be displayed in the value display section 183, and the setting values for each parameter of the parameter set are setting values that may be input (or changed) in the disclosure. after any one is selected from among at least two setting values comprising the setting value and one and more changed setting values the determination result of the determination process, to which the selected value has been applied, may be displayed.

The device according to the disclosure may display a recommendation range for the parameter value when the user (reagent researcher/developer) inputs the setting value. For example, when a cursor is present in the value-input area, it is possible to lead to the user's input to set the setting value in the recommendation range and allow the user to recognize a proper value for each parameter by displaying the recommendation range of the value pre-determined for the parameter.

The device according to the disclosure performs analysis according to the setting value of the setting value input parameter set selected by the user and displays the comparison item of the table 184 in the determination result display section 182.

As such, the device according to the disclosure may receive a user input to apply the per-parameter values of the pre-determined default value parameter set and per-parameter values of the setting value input parameter set selected from among the setting value parameter sets for comparison therewith, displayed in the value display section 183, to analysis and arrange and display the applied analysis result in the correct answer data of the table 184 displayed in the determination result display section 182, rendering it possible to reference the degree of the value of the predetermined default value parameter set in a single screen and, based thereupon, input a value for each parameter and immediately identify the input result.

Whether to display the pre-determined default value parameter set and setting value parameter set displayed in the value display section 183 in the section 183 may be selected by the user's selection. For example, only the setting value parameter set may be displayed, instead of the pre-determined default value parameter set. In this case, any setting value may be the default value, and the result of comparison between the setting values may be displayed in each of 186 and 187 in the determination result display section 182.

The positive/negative determination process has various parameters for which values should be set. However, it is typically not easy for one who is not a skilled developer to accurately set a value for each parameter used in the positive/negative determination process in the product development process.

Accordingly, the conventional real-time PCR reaction device displays the positive/negative determination result analyzed according to a pre-determined reference setting value for each parameter.

However, the developer is required to identify the positive/negative determination result while variously changing all or some setting values of the per-product parameters to find the optimal setting value for each product and identify what difference is present as compared with the actually identified positive/negative determination result. Further, during this course, the developer is also required to recognize the relationship between per-parameter setting values, changes in each parameter, and/or a specific parameter having a primary influence on positive/negative determination while changing the per-parameter setting values to reach the actually identified positive/negative determination result.

However, since the conventional real-time PCR reaction device displays only the positive/negative determination result analyzed according to the pre-determined reference setting value, it is difficult to simultaneously identify or compare the analysis results (positive/negative determination result) or changes (amplification curve) while variously changing the setting value.

In the disclosure, the user may identify a pre-determined default value in a single screen even without a separate screen shift, selectively screen the values for per-analysis type parameters by referring to the identified default value, perform a determination process, and identify the determination result. In other words, the disclosure may provide the determination result display section 182 and the parameter value display section 183 on the display 181 which is one screen as shown in FIG. 3, displaying the sections 182 and 183 on one screen.

This display type is merely an embodiment which is displayed on the display 181, one screen, according to the disclosure, but is not limited thereto. According to an embodiment, each of the sections 182 and 183 may be displayed on a dedicated window so that they may be displayed on one screen.

According to an embodiment, instead of the determination result table to which the default value and setting value each are applied, an image of the plate (or amplification curve) may be displayed in the determination result display section 182, and the determination result of the determination process may be displayed through identification information (e.g., red for positive, blue for negative, and gray for gray) about the well for each plate. In this case, when a value for each parameter is input in the setting value parameter set, the identification information or amplification curve may be changed according to the input setting value, and the correct answer data may be displayed as the number of positives/negatives or as a correct answer-applied plate image as described above.

Specifically, the determination process is performed using the result of reaction performed using the plate including a plurality of reaction wells containing the target analyte. The position of the reaction well different from the correct answer data among the first determination result of the determination process to which the default value is applied may be displayed on the imaged plate, and the position of the reaction well different from the correct answer data among the second determination results of the determination process to which the setting value is applied may be displayed on the imaged plate.

In this case, the reaction well different from the correct answer data means the result that the positive in the correct answer data is determined as negative or gray, and the negative in the correct answer data is determined as positive or gray.

According to an embodiment, since the result of nucleic acid amplification reaction is displayed as the plate image after the nucleic acid amplification reaction is performed, the user may easily select a plurality of reaction wells in the imaged plate. The reaction wells may be displayed in blue when the determination result is positive and in red when the determination result is negative. According to an embodiment, the reaction wells may be displayed in numbers, characters, or symbols.

In the default value-applied the first determination result of the determination process and the setting value-applied the second determination result of the determination process, the position of the reaction well different from the correct answer data may be displayed to be distinguished from the display of positive and negative on the imaged plate. For example, the difference from the correct answer data may be displayed in color, character, or symbol.

When a reaction well is selected from the imaged plate, amplification property data may be displayed. The amplification property data is a parameter used for the positive/negative determination process.

According to an embodiment, the amplification property data may be displayed in the reaction well different from the correct answer data. In other words, in the default value-applied the first determination result of the determination process and the setting value-applied the second determination result of the determination process, the amplification property data in the reaction well different from the correct answer data may be displayed.

According to an embodiment, the input setting value and the amplification property data may simultaneously be displayed on one screen.

Thus, since the experimenter or developer is able to intuitively distinguish reaction wells different from the correct answer data on the imaged plate, the parameter for the well of the corresponding plate (default value-applied or setting value-applied) may be reset for the changed value.

According to an embodiment, the step of displaying the amplification property data corresponding to the determination result different from the correct answer data of the default value-applied the first determination result of the determination process may be further included, or the amplification property data for the reaction well different from the correct answer data of the setting value-applied the second determination result of the determination process may be displayed.

According to an embodiment, the input default value and the amplification property data may simultaneously be displayed on one screen. Further, the input setting value and the amplification property data may simultaneously be displayed on one screen.

As shown in FIG. 3, per-parameter values of each of a pre-determined default value parameter set and setting value parameter set in the value display section 183 may be set for each of the high-temperature channel and low-temperature channel.

As shown in the value display section 183 of FIG. 3, the parameter setting interface providing device 100 according to the disclosure may selectively select a target analyte and a positive control group PC through an active/inactive window 192 in the default value parameter set and setting value parameter set displayed in the value display section 183. Accordingly, the default value and setting value for each parameter for the target analyte and the default value and setting value for each parameter for PC according to the disclosure may be separately set.

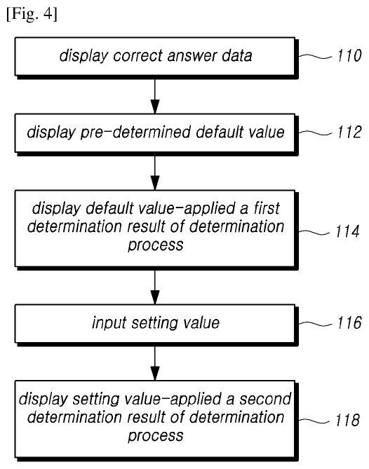

FIG. 4 is a flowchart illustrating a parameter setting interface providing method according to an embodiment. The stepwise operations of FIG. 4 are identical to the functions of the interface providing unit 180 of the parameter setting interface providing device 100, and thus, the description of FIGS. 1 and 2 may apply.

The above-described parameter setting interface providing device 100 is for providing an interface for setting a value for the Ct parameter operated as a positive/negative determination criterion and, according to an embodiment, perform analysis for a positive/negative determination process of a target analyte using the BPN (WO2017/086762) and DSP (WO2019/066572) technology as the positive/negative determination criterion. The BPN and DSP parameters may be applied to the functions provided by the parameter setting interface providing device 100 for setting the above-described Ct parameter value. For example, the BPN is a method for correcting and analyzing the data set and provides a normalization coefficient for correcting the data set obtained from signal-generation reaction for the target analyte using the signal generator. The normalization coefficient is provided using a data set including a plurality of data points including a reference value (the reference cycle), and cycles of signal generation reaction and signal values in the cycles. A cycle is selected between the cycles of the data set. The reference value is an arbitrarily determined value. The normalization coefficient is provided by determining the relationship between the reference value and the signal value of the cycle corresponding to the reference cycle in the data set. Corrected signal values are obtained by applying the normalization coefficient to the signal values of the data set, thereby providing a corrected data set and performing analysis.

The DSP obtains a data set for the target analyte from the signal-generation reaction using the signal generator to analyze the target analyte without false positive and false negative result, particularly false positive results, using the fitting accuracy of the nonlinear function for the data set as a direct indicator, corrects the obtained data set including a plurality of data points including cycle numbers and signal values, generates a non-linear function for the corrected data set to determine the fitting accuracy of the non-linear function for the corrected data set, and determines the presence or absence of the target analyte in the sample using the fitting accuracy. The analysis method requires various parameters and may be optimized according to the value set per parameter. The BPN (WO2017/086762) and DSP (WO2019/066572) technologies are used to assign parameters used in the presence/absence determination process for the target analyte and provide an interface for setting BPN and DSP parameter values.

The interface providing unit 180 may display pre-determined per-parameter values included in the BPN and DSP analysis methods. The disclosure provides an interface for displaying pre-determined default values of parameters for each analysis method of BPN and DSP.

Since the determination result of the determination process to which the predetermined default value is applied needs to match the correct answer data, the predetermined default value should be able to be previously set in the parameter setting interface providing device 100 or by a skilled experimenter based on the experimental result identified by an actual clinical experiment.

In the parameter setting interface providing device 100 of the disclosure, the parameters may be distinguished by the analysis method type, classified by the type, and displayed. Accordingly, pre-determined default values may also be distinguished and displayed by the parameter according to the analysis method types.

The parameters are used for mathematical processing on signals in the signal generation reaction that generates signals dependent upon the presence of the target analyte or used as a criterion for determining the presence or absence of the target analyte in the sample. Examples of values for determining whether the sample is positive/negative include the threshold cycle (Ct), delta Ct, melt Tm, melt peak, the threshold intensity value of the signal used to determine Ct, threshold to determine Ct; the fitting start cycle and final cycle used in removing background; when the algorithm disclosed in WO 2017/086762 is adopted, the reference value and the reference cycle; when the sigmoid fitting algorithm is used, R², the maximum slope of the sigmoid fitting curve, and difference between the maximum and minimum signal values; when the algorithms disclosed in WO2015-147370, WO 2015/147412, WO2015/147382 and WO2016/093619 are adopted, the reference value.

Thus, the BPN of the analysis method may include the reference value (RV) and signal value relation parameter (Cut-off ratio (CR)) value parameters. The CR specifies a change in signal, or relation or degree of signal change or signal difference, as a number, when two signal values occur at different detection temperatures. In this case, the different detection temperatures may be a relatively high detection temperature and a relatively low detection temperature, or a first temperature and a second temperature. “Signal value relation parameter” includes an arbitrary value reflecting the pattern (rule) of signal change at different detection temperatures. The signal value relation parameter is a value indicating the degree of change between a signal detected at a relatively high detection temperature and a signal detected at a relatively low detection temperature for a specific target nucleic acid sequence. The signal value relation parameter may indicate a value used to change, switch, adjust, or deform the signal detected at one temperature to a signal at another temperature. The signal value relation parameter may vary depending on the type of the target nucleic acid sequence, the type of the signal generator, and the conditions of incubation and detection. Accordingly, various signal value relation parameters may be determined for the same or different target nucleic acid sequences.

The signal value relation parameter may be represented in various aspects. For example, the signal value relation parameter may be represented as a numerical value, the presence/absence of signal, or a plot having a signal property.

The DSP includes parameters for correcting the data set obtained through signal generation reaction and parameters for number adjustment. The parameters for correcting the data set may include the parameter criterion (PMC) regarding determination of the shape of sigmoid graph for the data set obtained through signal generation reaction, start fitting cycle (SFC) for correcting the baseline fitting start cycle, and minimum fitting cycle (MFC) used for baseline fitting from the SFC to the minimum MFC to correct the data set.

The parameters for number adjustment may further include the cut-off ratio (CR) which is the value obtained by dividing the RFU value at the low temperature by the RFU value at the high temperature to correct the difference in REU value due to the difference in RFU value between low/high, threshold (Ct) for determining positive/negative after sigmoid fitting, determinant RFU (dRFU) for filtering the positive/negative of the sample, and R square criterion (RC) for processing as negative if R² is the R square criterion or less. U.S. Patent Application Publication No. 2009/0119020 may determine whether the data set indicates a significant or effective growth using the R² value of the sigmoid function and, in this case, if the R² value is the R square criterion or less, may further include the parameter RC indicating negative.

The parameter values may be set per detection channel (FAM, HEX, C610, Q670, or Q705) or per detection temperature.

The per-parameter values according to such analysis method type are values for determining positive/negative to converge to the positive/negative determination result identified in a clinical experiment.

As described above, when the BPN and DSP-applied default values are set in displaying the determination result of the determination process to which the default value for the Ct parameter is applied, the interface providing unit 180 may apply the default value set to the data generated in the reaction well of the plate (having undergone the nucleic acid amplification reaction) to be analyzed to perform analysis on a plurality of samples and display the determination result. In this case, the default value-applied determination result may be arranged and displayed with the number of positives, number of negatives, and gray (positive/negative) of the correct answer data.

As described above, the interface providing unit 180 may input each setting value for each parameter of the parameter set classified and displayed per analysis type for the setting value based on the BPN and DSP in inputting the setting value for the Ct parameter.

Referring to FIG. 3, the default value 189 is the parameter set of the BPN and DSP specified and displayed as the current setting value in the value display section 183, and the setting value is the parameter set of the BPN and DSP specified and displayed as the comparison setting value. The parameter set means a group of one or more parameters (e.g., RV and cut-off in BPN and PMC, SFC, MFC, THRD, and RPC in DSP) for each analysis method type according to the disclosure.

If a preparation space specified as a current setting value is displayed in the value display section 183, this is a default value for BPN and DSP and, as described above, the default value may be output, as a pre-determined fixed value, in the value display section 183 and displayed in the value display section 183, but may be a value that cannot be changed and is restricted for user access. The default value specified and displayed as the current setting value in the value display section 183 is a value regarding the determination result displayed as the reference item in the 184 table in the determination result display section 182. When the default value is displayed in the value display section 183, the device according to the disclosure may display the displayed default value-applied the first determination result of the determination process in the table (184) reference item in the determination result display section 182. The parameter set means a group of at least one or more parameters for each of at least one or more detection channels.

According to an embodiment, the parameter set is a parameter set for setting the per-parameter values for each detection channel.

The default value is a value for outputting the determination result of positive, negative, and gray (positive/negative), displayed in the reference item for each table in the determination result display section 182, and default values for Ct may be grouped and displayed per detection channel by specifying, e.g., it as the current setting value in the value display section 183.

Accordingly, the user may recognize the degree of the value for each parameter of the default value pre-determined for Ct which becomes a reference of the parameter used in the determination process. As described above, since the pre-determined default value is set by a skilled experimenter based on the experimental result identified by an actual clinical experiment so that the default value-applied the first determination result of the determination process matches the correct answer data.

If the parameter set specified as a comparison setting value in the value display section 183 is displayed, this is a setting value for Ct and, as described above, the setting value is a value that is selected and input by the user. The setting value may be previously input by the user and be previously stored with a specific name in the parameter set unit and be invoked and displayed in the value display section 183.

For the invoked and displayed setting value parameter set, a changed value for the setting value for each parameter may be newly input by the user in the value display section 183.

At least one or more setting value input parameter sets may be displayed in the value display section 183. In this case, the user may check any one setting value input parameter set among the setting value input parameter sets displayed in the value display section 183 and select it as a setting value input parameter set that may be compared with the correct answer data and the pre-determined default value-applied the first determination result.

As described above, the parameter setting interface providing method may provide an interface for setting Ct which is a parameter operating as a positive/negative determination criterion. When a correction task is performed on such signal, an interface used for setting the value of the parameter used for the correction task may be provided for the parameter.

When an element “comprises,” “includes,” or “has” another element, the element may further include, but rather than excluding, the other element, and the terms “comprise,” “include,” and “have” should be appreciated as not excluding the possibility of presence or adding one or more features, numbers, steps, operations, elements, parts, or combinations thereof. All the scientific and technical terms as used herein may be the same in meaning as those commonly appreciated by a skilled artisan in the art unless defined otherwise. It will be further understood that terms, such as those defined dictionaries, should be interpreted as having a meaning that is consistent with their meaning in the context of the relevant art and will not be interpreted in an idealized or overly formal sense unless expressly so defined herein.

The above-described embodiments are merely examples, and it will be appreciated by one of ordinary skill in the art various changes may be made thereto without departing from the scope of the disclosure. Accordingly, the embodiments set forth herein are provided for illustrative purposes, but not to limit the scope of the disclosure, and should be appreciated that the scope of the disclosure is not limited by the embodiments. The scope of the disclosure should be construed by the following claims, and all technical spirits within equivalents thereof should be interpreted to belong to the scope of the disclosure.

While embodiments of the disclosure have been described above, it will be apparent to one of ordinary skill in the art that the specific techniques are merely preferred embodiments and the scope of the disclosure is not limited thereto. Thus, the scope of the disclosure is defined by the appended claims and equivalents thereof.

### LEGEND OF REFERENCE NUMBERS

100: parameter setting interface providing device
120: communication unit
140: memory
160: processor
180: interface providing unit
200: detection device

### CROSS-REFERENCE TO RELATED APPLICATION

This application claims priority from Korean Patent Application No. 10-2022-0016907, filed on Feb. 9, 2022, in the Korean Intellectual Property Office, the disclosure of which is incorporated herein by reference in its entirety.

1. A method for providing an interface for setting parameters, for a target analyte positive/negative result-determination process; the method comprising:
displaying correct answer data for the positive/negative of the target analyte in a sample;
displaying a pre-determined default value for a parameter used in the determination process;
displaying a first determination result of the determination process to which the default value has been applied;
receiving a setting value for the parameter; and
displaying a second determination result of the determination process to which the setting value has been applied;
wherein the determination process analyzes a signal obtained by a reaction of amplifying a nucleic acid as the target analyte.

2. The method of claim 1, further comprising providing a section for displaying the first determination result and the second determination result,
wherein the first determination result and the second determination result are together displayed in the section for displaying the determination results.

3. The method of claim 2, wherein the correct answer data is displayed in the section for displaying the determination results.

4. The method of claim 1, further comprising providing a section for displaying a value of the parameter,
wherein the default value and the setting value are displayed in the section for displaying the value of the parameter.

5. The method of claim 1, further comprising:
receiving a changed value for the setting value; and
displaying the second determination result of the determination process to which the changed value has been applied.

6. The method of claim 1, further comprising: providing, on one screen, both (i) a section for displaying the first and second determination results of the determination process and (ii) a section for displaying the pre-determined default value and the setting value of the parameter.

7. The method of claim 5, further comprising displaying, on one screen, the setting value, the changed value and determination results by using the setting value and changed value.

8. The method of claim 5,
selecting any one of the at least two values comprising the setting value and one or more changed setting values; and
displaying the determination result of the determination process to which the selected value has been applied.

9. The method of claim 1, wherein the parameter is used in mathematical processing of the signal and/or is applied to the signal to be used as a criterion for determining the positive/negative of the target analyte in the sample.

10. The method of claim 9, wherein the criterion includes a threshold of the signal used to determine a threshold cycle (Ct) of the sample.

11. The method of claim 1, wherein the parameter comprises:
a reference value for correcting a signal dependent upon the presence of the target analyte in the sample;
a signal value relation parameter (cut-off ratio (CR)) reflecting a rule of the signal change at different detection temperatures;
a parameter criterion (PMC) regarding determination of a shape of a sigmoid graph for a data set obtained through the signal generation reaction;
a start fitting cycle (SFC) for correcting a baseline fitting start cycle;
a minimum fitting cycle (MFC) used in baseline fitting from the SFC to a minimum MFC to correct the data set; and
a determinant RFU (dRFU) regarding displacement of a data set obtained from the signal generation response for the positive/negative of the target analyte.

12. The method of claim 1, wherein the determination process is performed using a result of a reaction performed using a plate including a plurality of reaction wells containing the target analyte, and wherein the method further comprises:
displaying a position of a reaction well different from the correct answer data of the first determination result of the determination process to which the default value is applied, on an imaged plate; and
displaying a position of a reaction well different from the correct answer data of the second determination result of the determination process to which the setting value is applied, on an imaged plate.

13. The method of claim 12, further comprising displaying amplification property data when the reaction well is selected.

14. The method of claim 13, further comprising displaying the received setting value and the amplification property data on one screen.

15. The method of claim 1, further comprising:
displaying amplification property data corresponding to a determination result different from the correct answer data of the first determination result of the determination process to which the default value is applied; or
displaying amplification property data corresponding to a determination result different from the correct answer data of the second determination result of the second determination process to which the setting value is applied.

16. The method of claim 15, further comprising:
displaying the default value and the amplification property data on one screen; or
displaying the received setting value and the amplification property data on one screen.

17. A device for providing an interface for setting parameters, for a target analyte positive/negative result-determination process; the device comprising:
a memory;
a processor; and
an interface providing unit driven by the processor, wherein the interface providing unit is implemented to be driven by the processor executing instructions stored in the memory to:
display correct answer data for the positive/negative of the target analyte in a sample;
display a pre-determined default value for a parameter used in the determination process; display a first determination result of the determination process to which the default value has been applied;
receive a setting value for the parameter; and
display a second determination result of the determination process to which the setting value has been applied; wherein the determination process analyzes a signal obtained by a reaction of amplifying a nucleic acid as the target analyte.

18. A computer-readable recording medium storing a computer program including instructions that, when executed by one or more processors, enable the one or more processors to perform a method for providing an interface for setting parameters, for a target analyte positive/negative result-determination process; by a device for providing the interface for setting the parameters,
the method comprising:
displaying correct answer data for the positive/negative of a target analyte in a sample;
displaying a pre-determined default value for a parameter used in the determination process;
displaying a first determination result of the determination process to which the default value has been applied;
receiving a setting value for the parameter; and
displaying a second determination result of the determination process to which the setting value has been applied; wherein the determination process analyzes a signal obtained by a reaction of amplifying a nucleic acid as the target analyte.
